(19) United States
(12) Patent Application Publication
(10) Pub. No.: US 2006/0063202 A1
(43) Pub. Date: Mar. 23, 2006

(54) High throughput method and system for screening candidate compounds for activity against epilepsy and other neurological diseases

(75) Inventor: Vincent A. Pieribone, New Haven, CT (US)

Correspondence Address: Paul D. Greeley, Esq.;Ohlandt, Greeley, Ruggiero & Perle, L.L.P., 10th Floor, One Landmark Square, Stamford, CT, 06901-2682, US

(73) Assignee: John B. Pierce Laboratory

(21) Appl. No.: 11/201,575

(22) Filed: Aug. 11, 2005

Related U.S. Application Data
(60) Provisional application No. 60/600,493, filed on Aug. 11, 2004.

(51) Int. Cl. C40B 40/10; G01N 33/53; C12M 1/34

(52) U.S. Cl. 435/7.1; 435/287.2

(57) ABSTRACT

Methods and systems of compound screening are provided. Screening methods and instrumentation for candidate pharmacological agents are applied to discover compounds with particular activity against epilepsy. The method employs teleost fish, such as the medaka (Orzyias latipes), which are stimulated with a threshold electric field to produce convulsive behavior. The convulsive behavior is recorded optically and electrically. Antagonism of the convulsive behavior is produced by application of candidate pharmacological agents to the well containing the fish. The method can include stimulation and antagonism in a plurality of sample wells with a repetitive or simultaneous application of threshold electric fields. The methods and instrumentation can be applied to the study of other serious neurological diseases such as neuropathic pain.

This application claims priority from U.S. Provisional Application Ser. No. 60/600,493, filed Aug. 11, 2004.

### BACKGROUND OF THE INVENTION

1. Field of the Invention

The present invention relates generally to instrumentation and methods for screening putative pharmacological agents useful for the treatment of epilepsy in man and animals, using a model based upon electrical field threshold stimulation of medaka (Orzyias latipes) fish, contained in multiwell plates, and specifically in antagonizing the convulsive actions of the electric field threshold stimulation by the use of these pharmacological agents, as determined in the multiwell plates, wherein the convulsive response and its antagonism by the pharmacological agents is quantified by means of optical and electrical recording in the multiwell plates.

2. Description of the Related Art

Description of the Disease State and the Problem

Epilepsy was one of the first diseases against which drugs were effectively advanced. In 1857 the first anti-epileptic agent, bromide salt, appeared. In 1912 phenobarbital came into use and in 1938, phenytoin. The discovery and analoging of these drugs was aided early on by the development of the electroconvulsive-shock animal model of epilepsy. In the 1960s both carbamazepine and valproic acid were introduced and beginning in the 1990 there have been a range of new antiepileptic drugs introduced including felbamate, lamotrigine, gabapentin, topiramate and tiagabine.

Generally, the treatment of epilepsy has thus been considered to be a major success within neurology. However, this presumption is not completely correct. About one-half of newly diagnosed patients with epilepsy obtain complete control of their seizures with the first antiepileptic drug tried, and 13% more enter remission with a different (second) drug. The remainder of the population with epilepsy is not likely to obtain satisfactory seizure control with the use of any single drug or combination of multiple agents.

This vexing problem of pharmacoresistance is familiar to all clinical epileptologists. Many practitioners assume that certain epilepsies, such as catastrophic epilepsies of childhood and some lesional epilepsies including those associated with mesial temporal sclerosis and cortical dysgenesis, are more likely to be refractory to drug treatment, perhaps because the underlying mechanisms of seizure generation in these forms of epilepsy are especially resistant to antiepileptic drugs. However, in recent years it has become evident that diagnosis and organic pathology is a priori a poor predictor of whether an individual patient will respond to treatment with a specific drug or combination of drugs. Thus, there is a major unmet medical need for treatment of epilepsy, despite the major advances which have been made in pharmacotherapy within the last ten years.

Drug discovery has traditionally involved a number of methods for identifying novel chemical entities with activity against a particular disease process. Most commonly, a molecular target is identified which is involved in a disease, and compounds are then designed to interact with this target. Generally, the target is a receptor or enzyme, which can be examined in vitro and thus substantial numbers of compounds can be screened in an assay of some type.

Unfortunately, epilepsy is in fact a symptom represented by a spectrum of diseases arising from a range of etiologies, which derive from physiologic perturbations of the brain. In a minority of cases, gene mutations in a brain ion channel or receptor (“channelopathies”) have been identified. In some cases the onset of seizures is linked to some traumatic brain injury. In many instances the causes remain idiopathic. Therefore, establishing abnormally functioning biochemical targets can be difficult in most cases of this disease. However, several targets have been identified that if affected, reduce neuronal excitability and are therefore targets of epilepsy drug discovery. These include the excitatory and inhibitory amino acid neurotransmitter receptors. However, there are a number of pharmacological agents that are highly effective in treating various forms of the disease and yet the site of action of the compounds is either not clearly known, numerous (so called ‘dirty drugs’) and often with low affinity. Despite the fact that genetic screening has identified in animals protective genes from convulsions, there is not nor will there ever be a single target, which will modulate all epilepsies, due to the inherent heterogeneity of the disorders' etiology.

Animal Models of Epilepsy

There are various classical models of antiepileptic drug activity in rodents. Typically, one way to measure this is to assess a compound's ability to prevent the hind limb tonic extension component of the seizure in groups of mice induced by maximal electroshock (MES) after oral or intraperitoneal administration according to the procedures of the Epilepsy Branch, NINCDS as published by R. J. Porter, et at., Cleve. Clin. Quarterly 1984, 51, 293, and compared to the standard agents dilantin and phenobarbital. ED50 values are typically obtained. In this maximal electroshock seizure test (MES), corneal electrodes primed with a drop of electrolyte solution (0.9% NaCl) are applied to the eyes of the animal and an electrical stimulus (50 mA for mice, 150 mA for rats; 60 Hz) was delivered for 0.2 second at the time of the peak effect of the test compound. The animals were restrained by hand and released at the moment of stimulation in order to permit observation of the seizure. Abolition of hind-leg tonic-extensor component (hind-leg tonic extension does not exceed a 90° angle to the plane of the body) indicated that the compound prevented MES-induced seizure spread.

An alternative is the subcutaneous pentylenetetrazol threshold test (scMet), the convulsant dose (CD97) of pentylenetetrazol (85 mg/kg in rats) was injected at the time of peak effect of the test compound. The animals are isolated and observed for 30 minutes to see whether seizures occurred. Absence of clonic spasms persisting for at least five seconds indicated that the compound could elevate the pentylenetetrazol induced seizure threshold.

Clearly both of these rodent assays are not high throughput as extensive operator intervention is required. Thus 100s or 1000s of compounds can not be screened in a single day as is required in current drug discovery.

The present invention teaches methods and instrumentation for the determination of the efficacy of various pharmacological substances against a convulsive model of epilepsy. The approach utilizes Medaka (Orzyias latipes) fish as a medium to high-throughput screen for compounds with anticonvulsive properties. The present invention teaches the method, software and a proto-type device capable of monitoring and convulsing 48 fry simultaneously in the presence of classic anticonvulsants. An increased plurality of test wells such as a 96 well automated device for screening Medaka (Orzyias latipes) and the initial screening of small (10-50K chemical libraries) is further described.

Current drug discovery methodology demands the screening of large libraries of many thousands of chemical compounds. Using a mouse or rat model will not allow this to be done in a cost effective manner. Furthermore, it is highly undesirable to utilize large numbers of mammalian animals for such testing if there are alternatives due to ethical considerations. Accordingly, it is strongly desirable to develop an animal model that will allow at least a hundered-fold increase in the number of compounds that could be screened without increasing higher vertebrate animal use.

Epileptic activity in mammals is physiologically characterized by synchronized high voltage erratic EEG signals. The behavioral consequences of this activity are a range of uncontrollable actions or inactions. The behaviors range from violent muscular contractions and uncontrollable jerking to bouts of unconsciousness (absence). The type of seizures is highly dependent on the locus of its origin and the extent of its spread. Seizures can be of cortical or subcortical origin. Acute seizure activity can be stimulated in mammals by chemical and electrical stimuli.

A range of chemical agents can precipitate seizures. Such chemicals include picrotoxin, bicucline, fluroethyl, and strychnine. In general, these agents block the inhibitory neurotransmission via GABA or glycine, or enhance excitatory glutamate neurotransmission. Short duration (50-1000 ms) alternating electrical stimulation in a variety of waveforms applied to the cranium produce seizures in mammals. Chronic seizures can arise from subdural cranial injuries and a variety of other CNS insults. A variety of gene deletions have been shown to result in various forms of epilepsy. Finally, there are a number of idiopathic epilepsies that arise in humans.

Only a few studies have explored epileptogenesis in non-mammalian vertebrates. It is, however, clear that both the amphibia and teleosts CNS exhibit classical epileptiform activity. In spite of lacking any laminated cortical structures teleosts and amphibia do exhibit the electrical and behavior signatures of seizures. Penicillin has been shown to induce epileptiform electrical activity and behaviors in teleosts. Periods of spindly high-voltage low amplitude EEG characteristic of seizures can be illicted in teleosts by chemical stimulation. Electrofishing, which has been used for over 50 years and has been heavily studied by fishery scientists, can involve a process of applying an alternating pulse of high voltage to the water surrounding a fish. This procedure causes a short-lived period of heightened motor activity followed by a period of ‘sleep’ during which the fish are easily netted. This sequence appears to represent a ‘tonic/clonic-type’ seizure followed by an ‘absence-type’ seizure. In fact the full range of teleosts responses to electrical fields applied to water can be seen in mammals.

Because teleosts lack classic mammalian cortical structures, they do not provide a good model of cortical seizures. However, no significant differences appear to exist between the pharmacological and physiological profile of cortical and subcortical seizures. A notable exception to this rule is glycine-mediated seizures, which likely arise from the pervasive glycinergic system present in the spinal cord. Teleost have similar glycinergic mechanisms within the spinal cord.

Accordingly, the present invention seeks to use chemical and electrical initiated seizures in teleosts as a model of mammalian epilepsy.

### SUMMARY OF THE INVENTION

In broad concept, the present invention provides a high throughput screening system, which includes: a light source; a stimulation electrode array; a multiwell tissue culture plate; a photodetector; a stimulus isolation unit; and an A/D converter.

The present invention also provides a high throughput screening system, which includes:

a plurality of wells which form a multiwell tissue culture plate, each well being capable of containing one or more sample;

a pair of inert, conductive electrodes for applying a threshold electric field to the wells to produce convulsive behavior in the sample;

a plurality of photoemitters situated above each well of the multiwell tissue culture plate; and

an array of photodetectors situated below each well for recording the convulsive behavior in real time;

wherein certain wells of the multiwell tissue culture plate can contain one or more chemical compounds which serve as candidate pharmacological agents for antagonizing the electrically induced convulsive behavior in the sample by pharmacological action.

The present invention further provides a high throughput screening system, which includes:

a plurality of wells which form a multiwell tissue culture plate, each well being capable of containing one or more sample;

a plurality of photoemitters situated above each well of the multiwell tissue culture plate; and

an array of photodetectors situated below each well for recording normal rythym of the sample's beating heart in real time;

wherein certain wells of the multiwell tissue culture plate can contain one or more chemical compounds which serve as candidate cardiac pharmacological agents.

The present invention still further provides a high throughput screening system, which includes:

a plurality of wells which form a multiwell tissue culture plate, each well being capable of containing one or more sample;

a laser for producing a laser beam of appropriate wavelength for applying a threshold pain stimulation to the sample to produce an aversive behavior response;

a plurality of photoemitters situated above each well of the multiwell tissue culture plate; and

an array of photodetectors situated below each well for recording the response to aversive behavior in real time;

wherein certain wells of the multiwell tissue culture plate can contain one or more chemical compounds which serve as candidate pharmacological agents for antagonizing nocioceptive behavior in the sample or ameliorating the nocioception.

The present invention further still provides a method of screening a candidate pharmacological agent, which includes the steps of:

placing the candidate pharmacological agent in a multiwell tissue culture plate having a plurality of wells, each well being capable of containing one or more sample, wherein certain wells of the multiwell tissue culture plate can contain one or more chemical compounds;

applying a stimulus onto the sample to produce a change in the behavior of the sample;

recording the change in behavior in real time to select candidates with superior performance.

The present invention provides substantial time and efficiency advantages over prior techniques. The present invention can be used particularly advantageously in the industrialization of drug discovery processes. The present invention increases speed and productivity while providing researchers with expended capabilities and quality assurance.

### BRIEF DESCRIPTION OF THE DRAWINGS

FIG. 1 is a schematic representation of a high throughput screening system having a light source; a stimulation electrode array; a multiwell tissue culture plate; a photodetector; a stimulus isolation unit; and an A/D converter.

FIG. 2a depicts two fish in a well, wherein the fish are exposed to a brief electrical stimulation.

FIG. 2b is a graphical representation of the animals movements.

FIG. 3a shows the organization of the 21 pill photodiodes as they appear on the top of the photolithography board.

FIG. 3b shows one possible configuration in which photolithographic pads are produced which are positioned below the pill photodiodes to contact the bottom pole (usually the anode) of each element.

FIG. 3c shows a composite of the upper photolithographic electrode contacts super imposed on the diode array built on PC board.

FIG. 3d shows the position of the top anode contact superimposed on the diode arrays. The upper contact connects to the tab on each element.

FIG. 4a shows the timing sequence of valve openings (V1 and V2) in response to photodiode (PD) crossings.

FIG. 4b shows young fry that are concentrated in a fish reservoir.

### DETAILED DESCRIPTION OF THE PREFERRED EMBODIMENTS

The following description is provided to enable any person normally skilled in the art to make and use the invention and sets forth the best modes contemplated by the inventors of carrying out their invention. Various modifications, however, will remain readily apparent to those skilled in the art, since the general principles of the present invention have been defined herein specifically to provide a high throughput method and system for screening candidate compounds for activity against epilepsy and other neurological diseases.

The nomenclature used herein and many of the computer, detection, chemistry and laboratory procedures described below are well known and commonly employed in the art. The techniques and procedures are generally performed according to conventional methods in the art and various general references. The contents of all publications and patent documents cited in this application are incorporated by reference in their entirety as fully set forth herein for all purposes.

It has been unexpectedly found that the convulsion in the Medaka (Orzyias latipes) closely resembles that in humans, despite the lack of cortical structure and major differences in brain architecture and anatomy. It has been further found that anticonvulsant drugs, which are active in human epilepsy attenuate seizures in medaka (Orzyias latipes) in a dose-dependent manner resembling that in Man.

This assay, materials, and instrumentation for the use of Medaka (Orzyias latipes) fish as an epilepsy model will be useful in discovering compounds which act upon a variety of ion channels and targets in attenuating seizures. Notable among these are sodium channels and GABA receptors. These are briefly described as the utility of these assays, methods, and instrumentation, in part, is addressed by the therapeutic spectrum of neurological diseases, in addition to epilepsy, which are mediated by the sodium channels and GABA receptors.

A high throughput screening system including: a plurality of wells in the form of a tissue culture plate, wherein each well contains one, two, or more Medaka Fish (Orzyias latipes), wherein a threshold electric field is applied to the well by a pair of inert, conductive electrodes to produce convulsive behavior in the medaka fish, wherein the convulsive behavior is recorded in real time using an array of photodetectors situated below each well, and a plurality of photoemitters situated above and/or below each well of the multiwell tissue culture plate, wherein certain wells of the multiwell tissue culture plate contain one or more chemical compounds or mixtures of chemical compounds, which serve to antagonize by pharmacological actions the convulsive behavior electrically induced in the fish, and wherein the chemical compounds or mixtures of chemical compounds serve as candidate pharmacological agents in the high throughput assay.

In one embodiment, such a method includes placing one or more fish fry in each of a plurality of sample wells. A candidate drug compound is added to at least one of the plurality of sample wells; and after a suitable length of time a threshold electric fields is applied so as to produce convulsions in the fish fry. Apparatus for high throughput screening is also provided.

In one specific embodiment, a plurality of wells having one or more fish fry per well are each provided with two electrodes. A power supply is connected to the electrodes; wherein the power supply and the electrodes are configured to apply a series of electric fields to the fish fry, inducing convulsions, an optical detector is configured to detect light transmitted or refracted from the wells through the high transmittance portion, and a signal processing system is provided to interpret the light transmitted or refracted from the wells.

GABA System

A major target of the antileptic drugs is the GABA system. It has long been known that gamma aminobutyric acid (GABA) and glutamic acid are two major neurotransmitters involved in the regulation of brain neuronal activity. GABA is the major inhibitory neurotransmitter and L-glutamic acid is an excitatory. An imbalance in the concentration of these neurotransmitters can lead to convulsive states. Accordingly, it is clinically relevant to be able to control convulsive states by controlling the metabolism of this neurotransmitter.

When the concentration of GABA diminishes below a threshold level in the brain, convulsions result (Karlsson A, et al, Biochem. Pharmacol 1974;23:3053-3061). When the GABA levels rise in the brain during convulsions, seizures terminate (Hayashi T J, Physiol. (London) 1959;145:570-578). When GABA is injected into the brain of a convulsing animal, the convulsions cease (Purpura D P, et al, Neurochem. 1959;3:238-268). However, if GABA is administered systematically, there is no anticonvulsant effect because GABA, under normal circumstances, cannot cross the blood brain barrier (Meldrum B S, et al, Epilepsy; Harris P, Mawdsley C, eds., Churchill Livingston: Edinburg 1974:55).

Receptors for γ-aminobutyric acid (GABA), GABA_{A} receptors are the most abundant inhibitory receptors in mammalian brain. The GABA_{A} receptor are structurally constituted as macromolecular heteropentameric assemblies (combinations of α, β, and γ/δ protein subunits). Several subtypes of such GABA_{A} receptors have been described by techniques of modern molecular biology. Each GABA_{A} receptor complex includes a chloride ion channel that controls chloride flux across the neuronal membrane, and multiple recognition sites for small modulatory molecules such as benzodiazepines, barbiturates, picrotoxin, and certain steroids. When GABA interacts with its receptor, the ion channel is opened, chloride influx is enhanced, the membrane is hyperpolarized and the cell becomes less responsive to excitatory stimuli. This GABA induced ion current can be regulated by diverse agents. This receptor includes a multimeric protein of molecular size 230-270 kDa.

Agents that bind or interact with the modulatory sites on the GABA_{A} receptor complex, such as for example the benzodiazepine receptor, can have either enhancing effect on the action of GABA, i.e., a positive modulatory effect of the receptor (agonists, partial agonists), an attenuating effect on the action of GABA, i.e., negative modulation of the receptor (inverse agonists, partial inverse agonists), or they can block the effect of both agonists and inverse agonists by competitive block (antagonists or ligands without intrinsic activity). Agonists generally produce muscle relaxant, hypnotic, sedative, anxiolytic, and/or anticonvulsant effects, while inverse agonists produce proconvulsant, anti-inebriant, and anxiogenic effects. Partial agonists are characterized as compounds with anxiolytic effects but without or with reduced muscle relaxant, hypnotic and sedative effects, whereas partial inverse agonists are considered to be useful as cognition enhancers.

Such disorders include anxiety disorders, such as panic disorder with or without agoraphobia, agoraphobia without history of panic disorder, animal and other phobias including social phobias, obsessive-compulsive disorder, stress disorders including post-traumatic and acute stress disorder, and generalized or substance-induced anxiety disorder; neuroses; convulsions; migraine; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; and disorders of circadian rhythm, e.g., in subjects suffering from the effects of jet lag or shift work.

Further disorders for which selective ligands for GABA A receptors may be of benefit include pain and nociception; emesis, including acute, delayed and anticipatory emesis, in particular emesis induced by chemotherapy or radiation, as well as post-operative nausea and vomiting; eating disorders including anorexia nervosa and bulimia nervosa; premenstrual syndrome; muscle spasm or spasticity, e.g., in paraplegic patients; and hearing loss. Selective ligands for GABA A receptors may also be effective as pre-medication prior to anaesthesia or minor procedures such as endoscopy, including gastric endoscopy.

The present assay, methods, and instrumentation will there potentially discover pharmacological agents which, in addition to epilepsy, may be useful in ameliorating one or more of these various disorders supra in which GABA channels play an etiologic role.

Sodium Channels

Other major targets of antiepileptic drugs are voltage dependent sodium channels. Voltage-gated sodium channels are transmembrane proteins that cause sodium ions to flow through the membrane. Depolarization of the plasma membrane causes sodium channels to open allowing sodium ions to enter along the electrochemical gradient creating an action potential.

Such voltage-gated sodium channels are expressed by all electrically excitable cells, where they play an essential role in action potential propagation. They include a major subunit of about 2000 amino acids which is divided into four domains (D1-D4), each of which contains 6 membrane-spanning regions (S1-S6). The alpha-subunit is usually associated with 2 smaller subunits (beta-1 and beta-2) that influence the gating kinetics of the channel. These channels show remarkable ion selectivity, with little permeability to other monovalent or divalent cations.

Patch-clamp studies have shown that depolarisation leads to activation with a typical conductance of about 20 pS, reflecting ion movement at the rate of 10⁷ ions/second/channel. The channel inactivates within milliseconds.

Sodium channels have been pharmacologically characterised using toxins which bind to distinct sites on sodium channels. Channel blockers tetrodotoxin (TTX), a highly potent toxin from the puffer or Fugu fish, and saxitoxin (STX) bind to a site in the S5-S6 loop, whilst μ-conotoxin binds to an adjacent overlapping region. A number of toxins from sea anemones or scorpions binding at other sites alter the voltage-dependence of activation or inactivation.

Voltage-gated sodium channels that are blocked by nM concentrations of tetrodotoxin are known as tetrodotoxin sensitive sodium while sodium channels that are blocked by concentrations >1 uM are known as tetrodotoxin-insensitive (TTXi) sodium channels. TTX-insensitive action potentials were first studied in rat skeletal muscle, but are present as well in peripheral and central nervous tissue. Dorsal root ganglion neurons express at least three types of sodium channels, which differ in kinetics and sensitivity to TTX. Neurons with small-diameter cell bodies and unmyelinated axons (C-fibers) include most of the nociceptor (damage-sensing) population and express a fast TTX-sensitive current and a slower TTX-insensitive current.

Of the five cloned sodium channel a-subunit transcripts known to be present in dorsal root ganglia, none exhibits the properties of the TTX-insensitive channel. However, TTX-resistant sodium currents have been measured in rat nodose and petrosal ganglia. Sodium channels in peripheral nerve tissue play a large role in the transmission of nerve impulses, and therefore are instrumental in understanding neuropathic pain transmission.

Neuropathic pain falls into two categories: allodynia, where a normally non-painful stimulus becomes painful, and hyperalgesia, where a usually normal painful stimulus becomes extremely painful. The ability to inhibit the activity of these sodium channels, i.e., reduce the conduction of nerve impulses, will affect the nerve's ability to transmit pain. Selective inhibition of sodium channels in sensory neurons such as dorsal root ganglia will allow the blockage of pain impulses without complicating side effects caused by inhibition of sodium channels in other tissues such as brain and heart. In addition, certain diseases are caused by sodium channels that produce impulses at an extremely high frequency.

The ability to reduce the activity of the channel can then eliminate or alleviate the disease. Therefore, sodium channel blockers are used clinically to provide pain relief. Three classes of sodium channel blockers in common clinical use are local anesthetics such as lidocaine, some anticonvulsants such as phenytoin and carbamazepine, and some antiarrhythmics such as mexiletine. Each of these is known to suppress ectopic peripheral nervous system discharge in experimental preparations and to provide relief in a broad range of clinical neuropathic conditions.

The present assay, methods, and instrumentation will therefore discover pharmacological agents which, in addition to epilepsy, may be potentially useful in ameliorating one or more of these various disorders supra in which various types of sodium channels play an etiologic role.

The present invention can be used particularly in the industrialization of drug discovery processes. The present invention increases speed and productivity while providing researchers with expended capabilities and assuring quality. The present invention provides substantial time and efficiency advantages over prior techniques.

Definitions

The term “electrical stimulation” means initiating a voltage change in well containing a teleost fish fry, wherein such a voltage change produces a voltage in cells of the fish central nervous system resulting in a convulsion.

The term “threshold electrical stimulation” means initiating a voltage change in well containing a teleost fish fry, wherein such a voltage change produces a voltage in cells of the fish central nervous system resulting in a convulsion, wherein the voltage is the minimal required to produce a convulsion.

The term “electrode” means a controllable conductive interface between an instrument and a test system.

The term “Multiwell plate” refers to a two dimensional array of addressable wells located on a substantially flat surface. Multiwell plates can include any number of discrete addressable wells, and include addressable wells of any width or depth. Common examples of multiwell plates include 96 well plates, 384 well plates and 3456 well Nanoplates®. Such multiwell plates can be constructed of plastic, glass, or any essentially electrically nonconductive material

The term “A to D” Means analog to digital.

The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination.

The term “hit” refers to a test compound that shows desired properties in an assay.

The term “repetitive” means to repeat at least twice.

The term “resting potential” for a cell means the equilibrium transmembrane potential of a cell when not subjected to external influences.

The term “substantially parallel” means that the distance between the surfaces of two objects facing each other varies by less than 10%, preferably less than 5%, when measured at every point on the relevant surface of each object.

The term “test compound” refers to a chemical to be tested by one or more screening method(s) of the invention as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The term “transgenic” is used to describe an organism that includes exogenous genetic material within all of its cells. The term includes any organism whose genome has been altered by in vitro manipulation of the early embryo or fertilized egg or by any transgenic technology to induce a specific gene knockout.

The term “transgene” refers to any piece of DNA which is inserted by artifice into a cell, and becomes part of the genome of the organism (i.e., either stably integrated or as a stable extrachromosomal element) which develops from that cell. Such a transgene can include a gene which is partly or entirely heterologous (i.e., foreign) to the transgenic organism, or may represent a gene homologous to an endogenous gene of the organism. Included within this definition is a transgene created by the providing of an RNA sequence that is transcribed into DNA and then incorporated into the genome. The transgenes of the invention include DNA sequences that encode the fluorescent or bioluminescent protein that may be expressed in a transgenic non-human animal.

The term “transistor-transistor logic” or “TTL” refers to an electronic logic system in which a voltage around +5V is TRUE and a voltage around 0V is FALSE.

A “uniform electric field” means that the electric field varies by no more than 15% from the mean intensity within the area of observation at any one time.

Since the list of technical and scientific terms cannot be all encompassing, any undefined terms shall be construed to have the same meaning as is commonly understood by one of skill in the art to which this invention belongs. Furthermore, the singular forms “a”, “an” and “the” include plural referents unless the context clearly dictates otherwise. For example, reference to a “restriction enzyme” or a “high fidelity enzyme” can include mixtures of such enzymes and any other enzymes fitting the stated criteria, or reference to the method includes reference to one or more methods for obtaining cDNA sequences which will be known to those skilled in the art or will become known to them upon reading this specification.

These aspects of the invention and others described herein, can be achieved by using the methods and instrumentation described herein. To gain a full appreciation of the scope of the invention, it will be further recognized that various aspects of the invention can be combined to make desirable embodiments of the invention. Such combinations result in particularly useful and robust embodiments of the invention.

In the drawings, like reference numerals are used to identify identical components in the various views.

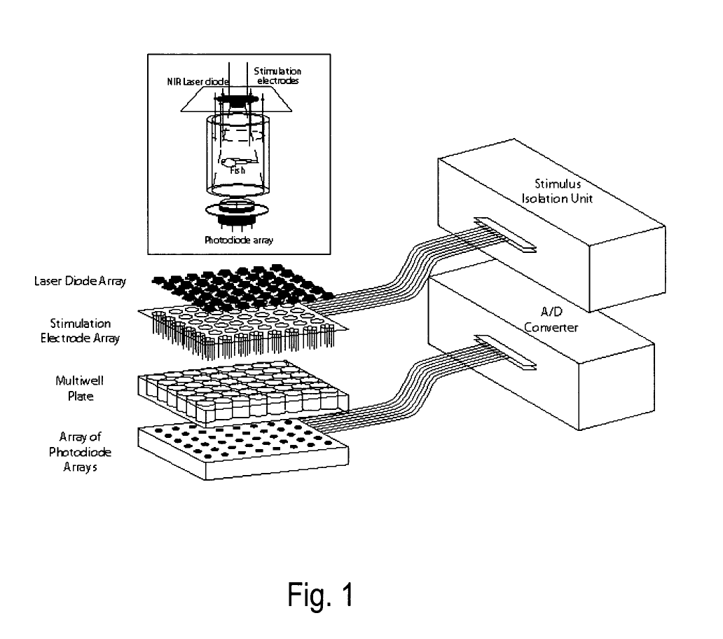

Referring to the Figures, FIG. 1 depicts a high throughput screening system having a light source, such as, a laser diode array, a stimulation electrode array, a multiwell tissue culture plate, a photodetector, such as, an array of photodiode arrays, a stimulus isolation unit and an A/D converter.

The screening system is a device designed to simultaneously stimulate and record the seizure activity of 48 teleost fish. Each well consists of a well containing tank water (100-1000 ml) and either a single or multiple young teleost (5 days-3 weeks).

The dish is maintained in a dimly lit or dark room. The temperature and sound level of the room can be maintained. On the bottom of each well is an array of photodiode arrays. Each well has an array of between 1 and 125 discrete photosensitive elements (photodiodes in this case but can be photovoltaics, phototransistors, photodarlingtons, CCD etc). The signal from each diode array is converted to digital information by a standard A/D converter and then fed to an array of computers. Multiple A/D converters and computers can be employed.

To illuminate the well a variety of forms have been employed, descrete Light Emitting Diodes (LED), arrays of LEDs, laser diodes, lasers and tungsten and fluorescent blubs. Infrared light is preferred although light of all wavelengths in the visible range has been tested. Into each well is a metallic electrode. These can consist of wire electrodes, or any form of metal (surface mount, photoconduct, etc.). Each well contains a positive and a negative (anode and cathode) terminal. These terminals exist as a set of two or as a plurality. Multiple electrodes are used in the preferred embodiment to produce a more even electrical field.

Electrodes can also be used on the bottom of the well as photoconduct on the well bottom. Electrical current is applied to each well from a stimulus isolation unit. This current can be applied as a constant current, constant voltage or constant power. Current can be applied in steps, ramps, sinusoidal, triangular, square waves and randomly. The frequency and duration can vary from 1-500 hertz and 1 ms to 1 second.

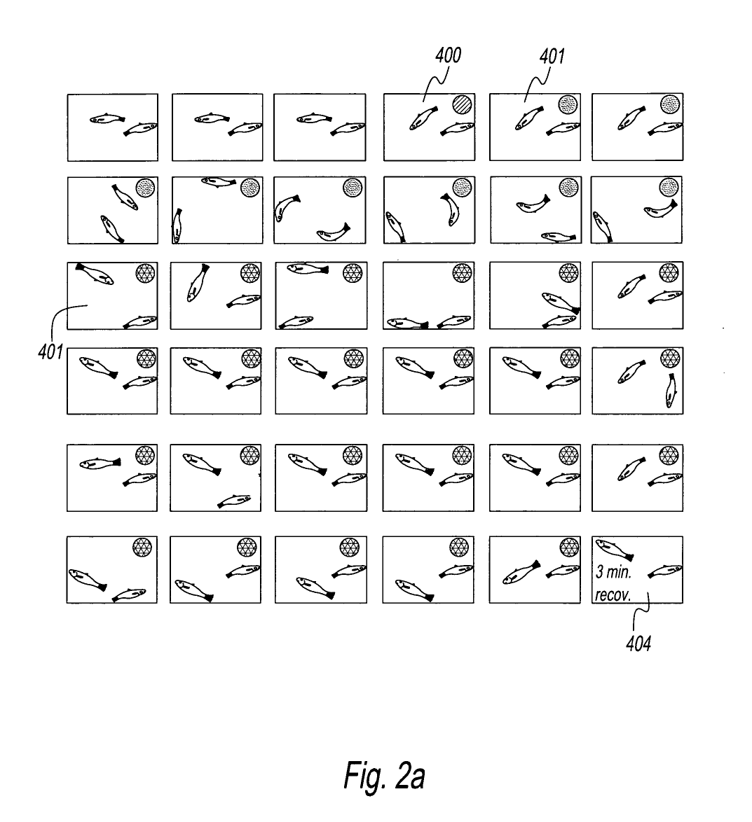

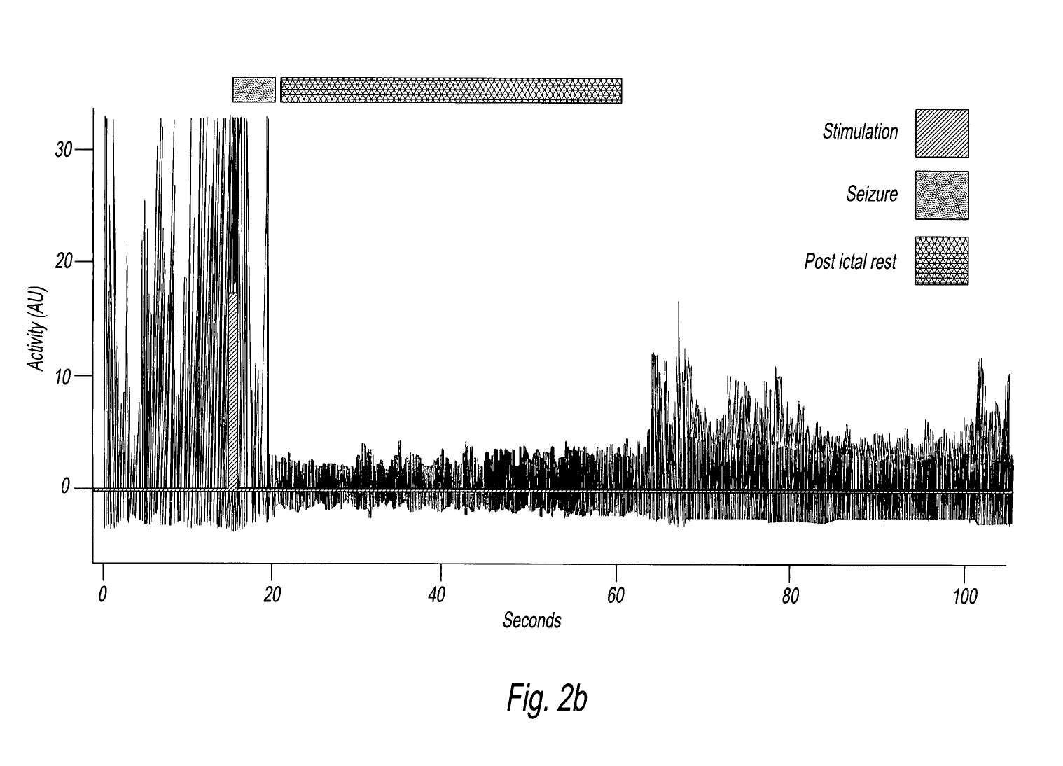

Referring to the FIGS. 2a and 2b, a typical experiment is described using electricity to evoke a seizure in a medaka fish.

FIG. 2a depicts two fish in a well, wherein the fish are exposed to a brief (100 ms, 60 hz, 0.1 mA) electrical stimulation (400) which is followed by a period of seizure (401-402) in which the fish exhibits erratic and heightened activity. This seizure period is followed by a period of reduced activity (402-404) termed the post-ictal rest period. This period is followed by a full recovery where the fish swim about the well as before (404).

FIG. 2b is a graphical representation of the movements of the animals. Optical signal obtained from the averaging of activity across 11 photodiodes positions below the well. An infrared diode laser was used to illuminate the well. The bottom trace is a magnification of a portion of the upper trace surrounding the electrical stimulation. The grey trace is the animal's activity and the black trace is the application of electrical stimulation.

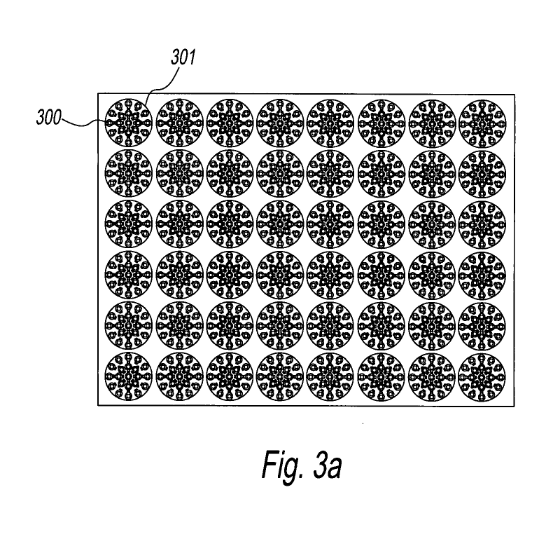

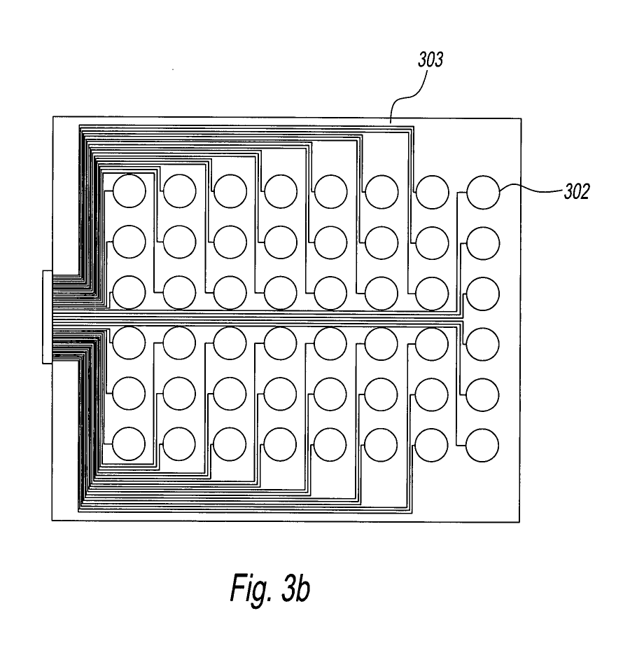

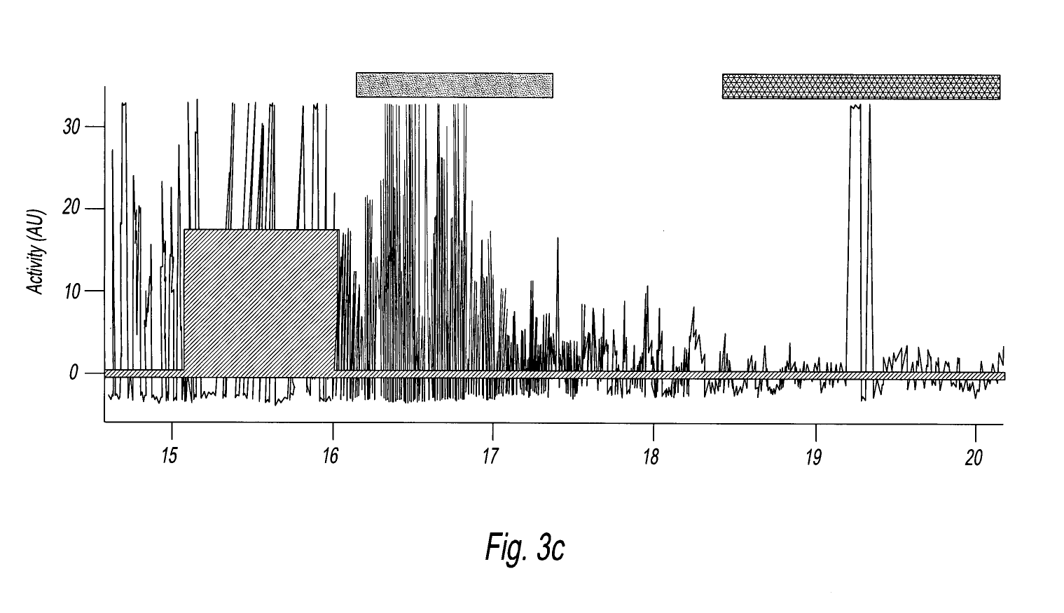

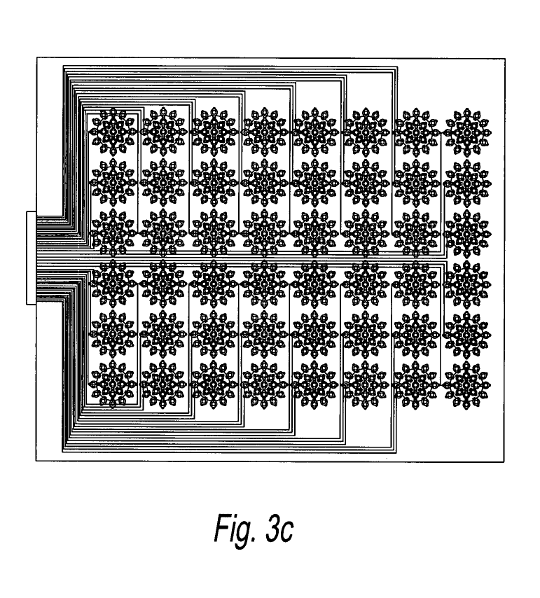

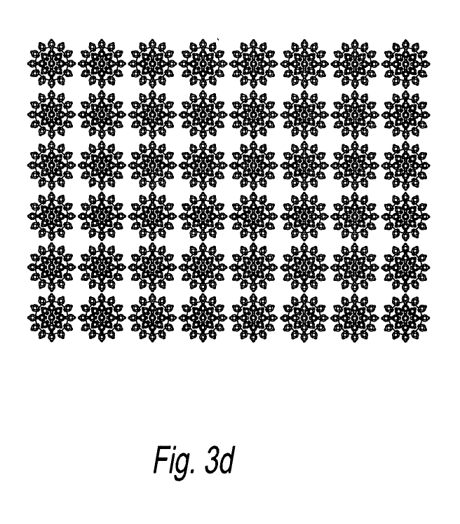

FIGS. 3a-d show a design for a 48-well photodiode array. Photolithography of a large substrate produces 48 arrays of 21 photodiodes (300). In this embodiment pill photodiodes are positioned in double-sided photolithography board. In one configuration all of the anodes from one well can be wired together as well as the cathodes from each well. In another configuration each discrete element is monitored separately.

FIG. 3a shows the organization of the 21 pill photodiodes as they appear on the top of the photolithography board. The large circles indicate the location of each well of a 48-well microliter plate. A plate is placed on top of the diode array so that each well has 21 diodes to detect fish motion. Devices with much higher density sensor elements can be employed. This can include CCD cameras, CCD chips, CMOS cameras, CMOS chips, fiber optic arrays and photolithography applied photosensitive elements.

FIG. 3b is a photonegative of PC-board traces and emitter contacts over photodiode arrays. One possible configuration is shown in which photolithographic pads which are positioned below the pill photodiodes to contact the bottom pole (usually the anode) of each element are produced. In this configuration all of the diodes on one well are summed. This is not the preferred embodiment, addressing each discrete diode is the preferred embodiment. Black lines indicate the traces that collect of the left side to be attached to a connector.

FIG. 3c shows PC-board traces and emitter contacts over photodiode arrays. A composite of the upper photolithographic electrode contacts super imposed on the diode array built on PC board is shown.

FIG. 3d shows emitter contacts over photodiode arrays. The position of the top anode contact superimposed on the diode arrays is shown. The upper contact connects to the tab on each element.

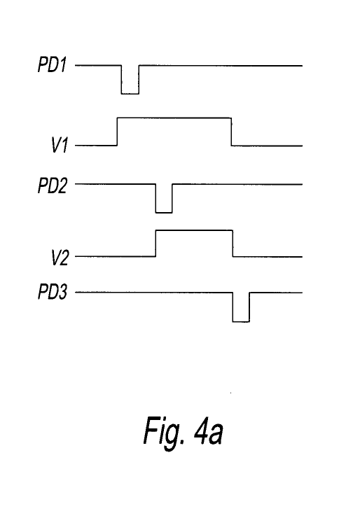

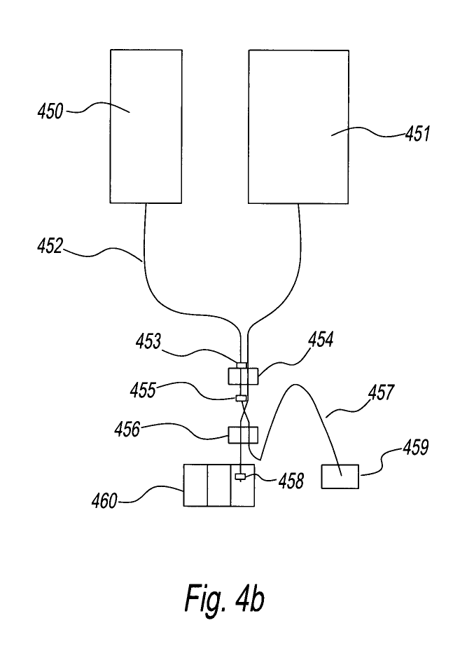

FIG. 4a shows the timing sequence of valve openings (V1 and V2) in response to photodiode (PD) crossings.

FIG. 4b shows young fry that are concentrated in a fish reservoir (450). Valve 1 (454) is opened to allow flow from the fish reservoir and V2 (456) is switched to allow water to flow into the waste reservoir (459). When a fish passes photodiode 1 (453) it produces a transient block of the light and reduction in the PD voltage. This results in the switching of valve 1 to open flow from the tank water reservoir (451) and close flow from the fish reservoir (450). The fish is then drawn down and passes PD2 (455) which switches V2 (456) from flow into the waste reservoir and into the well. When PD3 (458) is activated all valves return to the start position and the plate is moved to the next well.

1. Selection of Animals

Two species of teleost fish can be used; zebra fish (Brachydanio rerio) and the Japanese rice fish Medaka (Orzyias latipes) (Oryzias latipes). Zebra fish are familiar to many U.S. scientists but Medaka (Orzyias latipes) is a teleost model developed in Japan. Both systems have distinct advantages. The Medaka (Orzyias latipes) offers a complete genomic sequence. However, no doubt the full sequence of zebra fish will soon be available. There exists an almost completely transparent strain of Medaka (Orzyias latipes). A similar such strain exists for zebra fish although not quite as amendable.

Zebra fish have the distinct advantage of reproducing at a much faster rate. Female zebra fish can lay between 200-400 eggs at a time. Medaka (Orzyias latipes) only produce around 7-20 at a time. Zebra fish eggs hatch on a much quicker and more consistent schedule, 40-48 hours after being layed. Medaka (Orzyias latipes) take anywhere from 6-8 days. However, Medaka (Orzyias latipes) emerge in a much more mature state. Zebra fish have more difficult feeding requirements as fry. Medaka (Orzyias latipes) can be supported immediately upon birth on brine shrimp.

It is important to assure that all animals are precisely the same age and have been exposed to an identical physiological environment. The animals are highly responsive to light and are maintained on a constant 12/12 hr light-dark cycle. Temperature of the aquaria is maintained at 40° F. with a chiller. Well-water (non-chlorinated) is used to propagate the fish and to raise them. Temperature, pH, and ionic composition of the well water are monitored continuously.

2. Physiological Monitoring

There are two behavioral responses that can be measured as indicative of a seizure. First are rapid and erratic tail movements that accompany seizures and second is the post-ictal inactivity period. This approach can allow measurement and quantification of a frys' behavior during and following a seizure. This post ictal rest period is reproducible in duration and indicative that a seizure has occurred. Agents that block the production of a seizure produce a very active fish following stimulation that does not show typical seizure activity and does not exhibit the post-ictal somulance.

An optical approach entails the use of a light beam between an LED emitter and detector pairs which is disrupted by the animals movement in the well. Good signals can be obtained with either reflectance emitter detector modules (situated on the bottom of the well) or transmittance modules passing through the wells. It is important to use light that is not visible to fish in order to monitor the animal's motions. We initially used visible light to the fish and found that it was not possible to quantify the behaviors following the convulsion with this method. It appears that teleosts are able to see light up to around 670 nm and have a 570 nm centered rhodopsin and yet appear to be very sensitive down into the ultraviolet. Given the dangers of ultraviolet light and the difficulties in producing and detecting it for the purposes of the current experiments, we chose near infrared (NIR) light.

There are a number of different standard diode packages with emission wavelengths in the NIR. They range in wavelengths from 650-900 nm. It was found that at wavelengths above 800 nm, while good signals could still be obtained the fishes tail becomes almost transparent to this wavelength range of light, making the subtle movement less detectable. Therefore it was found that light in the 700-800 nm range was optimal. Transmitted rather than reflectance detection was found, unexpectedly, to be more sensitive. It was also determined that collimated and highly parallel light produced better ‘shadowing’ of the animal on the detector producing greater contrast and therefore larger movement signals. Thus, it was found to be optimal to employ NIR laser diodes as the emitters for each well. Such emitters contained in variable intensity packages including a culmination lens can be easily obtained.

In principle, the illumination could be provided by any of a variety of light sources, such as, incandescent lamps, hollow cathode lamps, gas vapor lamps, light emitting diodes, lasers, semiconductor diode lasers, tunable dye lasers, and the like. Desirably, the light source will provide for a time-varying light signal. For example, the intensity of light can be modulated electronically according to well known techniques to vary its output sinusoidally or in other patterns at a determined frequency in the range of about 10 Hz to 100 kHz, usually 100 Hz-50 kHz, more usually 1-20 kHz, during the period of irradiation.

For detection, a number of detector options (i.e. photodiodes, photoresistors, photodarlingtons and phototransistors) were evaluated. It was determined that a small array (n=4) of photodiodes covering the bottom of the well provides the greatest signal. Each well contains a plurality of photodetectors, wherein the number of photodetectors is at least four, and is optionally six, eight, ten, twelve, fourteen, sixteen, eighteen, or twenty. Using discrete photodiodes the number of photodiodes is most optimally in the range of eight to twelve. However, photodiodes can be made in arrays to physically match each well. Using readily available 1.2-micron technology, photodiodes on a 20-micron grid could be made for a 10×10 array. This would give 100 photodiodes in an area only 0.04 mm², i.e., 2500 photodiodes per mm². Even more density is possible using 0.5-micron processes that are commercially available. It is likely that a 1000 element array will fit on a 5 mm×6 min IC, which would be considered a medium-sized die format.

The photodector has a photoresponsive electrode. This is preferably a semiconductor electrode from which an electrical signal is inducible or variable, depending upon the effect of irradiation. The photoresponsive electrode(s) will have a semiconductive material.

Semiconductive materials include such materials as silicon, gallium arsenide, gallium selenide, aluminum gallium arsenide, or the like. The semiconductive material will be either of the p- or n- type and, as appropriate, may be intrinsic or may employ such dopants as boron, aluminum, phosphorus, arsenic, antimony, and the like. The degree of doping can be varied widely, there being a wide variety of commercially available doped wafers that can be used. The concentration of the dopant will normally vary empirically in order to provide the desired photoresponse, frequently being a matter of convenience, and will generally range from about 10¹⁰ to 10²⁰ atoms/cc; usually for silicon the rating will be about 5-20 ohm-cm. Photoconductive materials include chlorogallium phthalocyanine, TTF-TCNQ, and others.

Where the intensity of the light source is modulated, the electronic signal derived from a photoresponsive electrode can be selectively detected or measured with synchronous frequency and/or phase detection techniques, frequency selected electronic filtering, gated amplifiers, or the like, according to known techniques.

Other methods of physiological monitoring can also be used. Such methods include:

monitoring of the fish tail movement, which is rapid, by means of a microphone or miniature piezoelectric sensor which is placed in each well;

monitoring of the magnetic field generated in the well by the fish, most particularly when the fish has been administered magnetic particles, which produce a large emectromagentic signal in the well;

monitoring of the magentoelectroencephalogram of the fish in the well; and

monitoring of the electrocardiographic pattern of the motion of the heart in the fish in the well.

3. Signal Processing

Optical Detector and Amplifier Circuit

The optical detector and amplifier circuit can be implemented on an integrated circuit in order to convert an optical signal into an electrical signal suitable for data digitization and capture by a computer.

The optical detector and amplifier includes a phototransistor coupled to a transimpedence amplifier, which converts a current signal into a voltage signal and which is followed by an amplifier. The operational amplifier in the transimpedence amplifier is a two-stage, unbuffered amplifier.

In one embodiment, the capacitor has a value of 2 pF and the resistor has a value of 100 KΩ. The gain of amplifier is equal to 1+(Resistance/Resistance). Thus, in one specific embodiment for which the gain of amplifier is 10, resistors and are chosen so that their ratio is 9. The t is designed to be useful for wide-band amplification and low-level signals. The gain-bandwidth product is 70 MHz, and the amplifier is stable for gains greater than 10. In addition, the circuit has an input offset voltage less than 5 mV, a DC gain of 220, a positive slew rate of 80 V/μs, and a negative slew rate of 9 V/μs. The circuit requires 2.5 mW from a single 5 V supply.

The circuit can be modified as required by specific applications. Since the phototransistor cell is composed of basic photocell elements, it can be connected to as many cells as needed to create a desired geometry or a required number of channels needed to adapt the detector to a specific application. Other light sensing structures in addition to the phototransistor can be fabricated using standard CMOS processing steps. Several photodiode structures are possible using standard p-n junctions.

Although this embodiment has illustrated one method for measuring low current levels from a phototransistor, many other methods are possible and could be used without departing from the spirit and scope of the present invention.

For example, one method of determining the current would be to integrate the current using an integrating amplifier for a fixed time and then measure the voltage or to integrate the current until a fixed voltage is reached and then to measure the time.

A second method to determine the current would be to use an oscillator. In particular, an integrator could continually integrate the unknown current until a preset a voltage was reached and then reset itself. The resulting frequency of oscillation would be proportional to the current. In addition to using standard capacitors, one could implement this second method using the capacitance of the photodiode itself.

Initially, the switch is closed and the capacitance of the photodiode is discharged and there is zero voltage across the photodiode. When the switch is opened, light impinging on the photodiode creates charge, which produces a voltage on the photodiode capacitance. More light increases the voltage, which is amplified by the amplifier.

When the output of the amplifier exceeds the reference voltage, the output of the comparator changes state, firing the one shot which, in turn, closes the switch and discharges the photodiode capacitance. This resets the amplifier input to the initial state and the comparator also returns to its initial state. When the one-shot times out, the switch opens so that the process of charging can start again. A greater light level results in faster charging and therefore a higher frequency output of the oscillator. This results in an array of integrators that could be multiplexed to a single fast amplifier without the loss of significant measurement time.

Signal processing utilizes two 64-channel A-to-D conversion device assembled from National Instruments hardware. This system will digitize two signals from each well, the activity signal and the application of either the chemical or electrical convulsant. Each of these signals is digitized at around 1000-2000 samples per second. Using LabView programming, activity within wells can be assayed prior to stimulation to ensure adequate activity. Upon reaching a point of adequate spontaneous activity, the system will automatically deliver the convulsant, and the nature and duration of the seizures and post ictal inactivity will be measured. The digitized s signal is thresholded and fed into a TTL one shot multivibrator, which feeds an audio monitor (speaker). We have found that being able to monitor the movements as sound allows us to compare a visible signal we see on a video monitor with the sound of the animals' movements.

Other, more sophisticated signal detection algorithms can be effectively employed to increase the signal to noise ratio. These include fast Fourier Transformation of the summed signals thus allowing conversion from the amplitude domain to the time domain; amplitude discrimination analysis using a software pulse height analysis system, which is a simple extension of the threshold detection system actually employed in obtaining the results supra; and a parallel processor based, trainable, neural network model for signal elucidation. All of these methods and implementation of the associated algorithms are well known to those normally skilled in the art of signal detection and extraction.

4. Final Device Prototype

The final prototyped device has a 96 well array of photodetectors, wherein each well contains a plurality of photodetectors, and wherein the number of photodetectors is at least four, and is optionally six, eight, ten, twelve, fourteen, sixteen, eighteen, or twenty, but in no case greater than 128. Most optimally the number of photodetectors is in the range of eight to twelve. This device contains the associated amplification and integration hardware in a single printed circuit board. The device integrally contains and supports a multiwell plate to which a second device containing a similar array of photo emitters and electrical stimulators can be secured atop of the plate in a sandwich. A plurality of such devices can be optionally employed as part of the present invention.

Other types of multiwell plates can be used, as for example containing 384 wells, 1096 wells, 48 wells, 24 wells, 16 wells, and so forth. The present examples are non-limiting with regard to the plurality of wells in the particular design. Larger number of wells would require smaller optical detection components but such components of reduced size are readily commercially available and can be constructed by one skilled in the art. For example, such components of reduced size are commonly used in the production of inexpensive digital cameras for the consumer market.

Methodologically, (I) a physiological baseline is determined for each well; (II) threshold electrical stimulation is applied to evoke a seizure from the fish(s) in each well; and (III) physiological monitoring and quantification of the seizure-related motion events is performed.

Electrodes and Electrode Arrays

In the present embodiment, the design includes electrodes, and electrode arrays, for creating electrical fields across the area of observation. Typically this is achieved by the use of a pair of electrically conductive electrodes. An important design feature is that the electrode pairs create well-defined electrical fields. Preferred electrode designs include electrode configurations that maximize the electric field homogeneity within the well.

Field uniformity over a fixed area can be described in two ways:

(1) the standard deviation of the field magnitude divided by the average field magnitude in the area; and

(2) the difference between the highest and lowest field magnitudes, normalized to the average field magnitude in the area.

The simplest way to generate a uniform electric field in a conductive medium is to use two identical, flat electrodes with surfaces that are aligned substantially parallel to each other. Typical round multiwell plate wells however limit the width of electrodes that can be inserted into the wells, and also introduce two other effects which reduce field uniformity. The roundness of the wells provides a challenge to create a uniform field pointing in one direction with two electrodes the width of the water between the electrodes is constantly changing. Additionally the high surface tension of water generates variations in the height of the saline across the well when dipper electrodes are inserted.

The curved surface, or meniscus, can perturb the electric field throughout the volume of the well. The depth of 100 μL of saline in a 96-well plate is normally about 3.0 mm deep at the center and about 2.9 mm deep at the edges of the well. In one aspect the present invention includes improved electrode designs, and systems for electrical stimulation that address these issues to create substantially uniform electrical fields over the area of observation.

In one embodiment the electrode pair includes two substantially parallel wire electrodes including an electrical insulator that is attached to the pair of electrodes to restrict current flow to a defined region thereby creating a highly uniform electrical field.

We have developed a protocol to produce stimulation of the well that involves rapidly switching amongst six electrodes placed around the perimeter of the well. This is similar to the scrambling electrode arrays used in rodent operant boxes. By rapidly (approximately 100 Hz) switching the active electrodes, the voltage field is randomized and therefore more similar from well to well. Finally if the animal contacts the metal of the electrodes the shock delivered is greater than that experienced when tank water is in between.

To reduce the likelihood that the animal contacts the wire electrodes, a variety of electrode shielding arrangements have been tested allowing electrical conductivity and yet shielding the animal from contact. The most preferred method of allowing shielding is to cover the electrodes with fine (>600 mesh) Teflon mesh cloth. This prevents physical contact of the fish with the wire electrodes.

Any electrically conductive material can be used as an electrode. Preferred electrode materials have many of the following properties: (1) they do not corrode in saline, (2) they do not produce or release toxic ions, (3) they are flexible and strong, (4) they are relatively inexpensive to fabricate, (5) they are non porous, and (6) they are easily cleaned.

Preferred materials include noble metals (including gold, platinum, and palladium), refractory metals (including titanium, tungsten, molybdenum, and iridium), corrosion-resistant alloys (including stainless steel) and carbon or other organic conductors (including graphite various conductive polymeric materials). In most cases, stainless steel provides a preferred electrode material. This material is inexpensive, easy to machine, and very inert in saline. Stainless steel oxidizes slowly to produce iron oxide when passing current in saline, but this does not appear to affect the performance of the system. Iron oxide has very low solubility in water and toxic levels of iron do not appear to be released. Additionally any iron oxide deposits can easily be removed by soaking the electrodes in 10% nitric acid in water for two hours, then rinsing thoroughly with distilled water.

Electrolysis products can be contained or eliminated by coating the surfaces of the electrodes with protective coatings, such as gelatin, polyacrylimide, or agarose gels chloride electrode. Dipper electrodes typically includes one or more pairs of electrodes that are arranged in an array that can be retractably moved into, and out of, one or more wells of a multiwell plate.

Dipper electrodes can be orientated into arrays that match the plate format and density, but can be in arrays of any configuration or orientation. For example, for a standard 96 well plate, a number of electrode configurations are possible including electrode array arrangements to selectively excite one or more columns, or rows, simultaneously. The entire array of electrodes is held in correct registration by a rigid non conductive member that keeps each electrode pair correctly spaced to accurately match a standard 96 well plate layout.

The non-conductive member provides for the electrodes to move up or down while precisely maintaining their registration with the multiwell plate.

To provide for correct registration of the electrode array with a multiwell plate, the electrode assembly can optionally include an outer border or flange that can accommodate a standard 96-well plate, and enables accurate plate registration. In some embodiments, the border can further include a registration notch or indentation to provide unambiguous plate registration.

In a preferred embodiment the electrode array further includes means for retractably inserting the electrode array into the wells of the multiwell plate. In one configuration, the electrode array further includes an upper, movable support member to which the electrodes are attached. The movable support member is able to move up or down relative to the non-conductive member by sliding on a plurality of alignment pins. A pneumatic cylinder enables the movable support layer to automatically return to the upper position.

Multiwell Plates

Tissue culture assemblies, commonly referred to as tissue culture plates or multiwell plates, are used for in vitro cultivation of cells particularly for experimental purposes. In order to increase throughput, multiwell tissue culture plates, which include six, twelve, twenty-four, forty-eight, ninety-six, three hundred eighty four and one thousand fifty hundred thirty-six wells have been designed. Such multiwell tissue culture plates allow the investigators to use semi- or fully-automated devices to conduct tests of many individual cell cultures at a speed unachievable by regular tissue culture assemblies.

Multiwell tissue culture assemblies are exemplified in U.S. Pat. Nos. 4,349,632; 4,038,149; 4,012,288; 4,010,078; 3,597,326 and 3,107,204. Another culture vessel is exemplified in U.S. Pat. No. 4,358,908. Still another system is exemplified in U.S. Pat. No. 4,657,867A1.

The multiwell plates of the present invention are designed primarily to provide for efficient electrical stimulation of cells while at the same time enabling the optical analysis of transmembrane potential changes. To accomplish this conductive surface, electrodes can be orientated in, or on, the walls, bottoms or lids of the multiwell plate. In general such multiwell plates can have a footprint of any shape or size, such as square, rectangular, circular, oblong, triangular, kidney, or other geometric or non-geometric shape. The footprint can have a shape that is substantially similar to the footprint of existing multiwell plates, such as, the standard 96-well microtiter plate, whose footprint is approximately 85.5 mm in width by 127.75 mm in length, or other sizes that represent a current or future industry. Multiwell plates of the present invention having this footprint can be compatible with robotics and instrumentation, such as multiwell plate translocators and readers as they are known in the art.

Typically, wells will be arranged in two-dimensional linear arrays on the multiwell plate. However, the wells can be provided in any type of array, such as geometric or non-geometric arrays. The multiwell plate can include any number of wells. Larger numbers of wells or increased well density can also be easily accommodated using the methods of the present invention. Commonly used numbers of wells include 6, 12, 96, 384, 1536, 3456, and 9600.

Well volumes typically vary depending on well depth and cross sectional area. Preferably, the well volume is between about 0.1 microliters and 500 microliters. Wells can be made in any cross sectional shape (in plan view) including, square, round, hexagonal, other geometric or non-geometric shapes, and combinations (intra-well and inter-well) thereof. Preferred are square or round wells, with flat bottoms.

The walls can be chamfered, e.g., having a draft angle. Preferably, the angle is between about 1 and 10 degrees, more preferably between about 2 and 8 degrees, and most preferable between about 3 and 5 degrees.

The wells can be placed in a configuration so that the well center-to well-center distance can be between about 0.5 millimeters and about 100 millimeters. The wells can be placed in any configuration, such as a linear-linear array, or geometric patterns, such as hexagonal patterns. The well-to-well distance can be about 9 mm for a 96 well plate. Smaller well-center to well-center distances are preferred for smaller volumes.

The wells can have a depth between about 0.5 and 100 millimeters. Preferably, the well depth is between about 1 millimeter and 100 millimeters, more preferably between about 2 millimeters and 50 millimeters, and most preferably between about 3 millimeters and 20 millimeters.

The wells can have a diameter (when the wells are circular) or maximal diagonal distance (when the wells are not circular) between about 0.2 and 100 millimeters. Preferably, the well diameter is between about 0.5 and 100 millimeters, more preferably between about 1 and 50 millimeters, and most preferably, between about 2 and 20 millimeters.

Each well also includes a bottom having a high transmittance portion. Preferably, the bottom is a plate or film as these terms are known in the art. The thickness of the bottom can vary depending on the overall properties required of the plate bottom that can be dictated by a particular application. Well bottom layers typically have a thickness between about 10 micrometers and about 1000 micrometers. Preferably, the well bottom has a thickness between about 10 micrometers and 450 micrometers, more preferably between about 15 micrometers and 300 micrometers, and most preferably between about 20 micrometers and 100 micrometers.

The bottom of a well can have a high transmittance portion, typically meaning that either all or a portion of the bottom of a well can transmit light. The bottom can have an optically opaque portion and a high transmittance portion of any shape, such as circular, square, rectangular, kidney shaped, polygonal, or other geometric or non-geometric shape or combinations thereof.

Preferably, the bottom of the multiwell plate can be substantially flat, e.g., having a surface texture between about 0.001 mm and 2 mm, preferably between about 0.01 mm and 0.1 mm (see, Surface Roughness, Waviness, and Lay, Am. Soc. of Mech. Eng., #ANSI ASME B46.1-2985 (1986)). If the bottom is not substantially flat, then the optical quality of the bottom and wells can decrease because of altered optical and physical properties of one or both.

The materials for manufacturing the multiwell plate will typically be polymeric, since these materials lend themselves to mass manufacturing techniques. However, other materials can be used to make the bottom of the multiwell plate, such as, glass or quartz.

The bottom can be made of the same or different materials and the bottom can include polystyrene, or another material. Preferably, polymers that have low fluorescence and or high transmittance are selected. Polymeric materials can particularly facilitate plate manufacture by molding methods known in the art and developed in the future, such as, insert or injection molding.

The multiwell plate of the present invention can be made of one or more pieces. For example, the plate and bottom can be made as one discrete piece. Alternatively, the plate can be one discrete piece, and the bottom can be a second discrete piece, which are combined to form a multiwell plate. In this instance, the plate and bottom can be attached to each other by sealing means, such as adhesives, sonic welding, heat welding, melting, insert injection molding or other means known in the art or later developed.

The plate and bottom can be made of the same or different material. For example, the plate can be made of a polymer, and the bottom made of polystyrene, cycloolefin, glass, or quartz.

Automatic Distribution of Fish to Multiwell Plate Wells

In order for this assay to be feasible, a large number of animals must be assayed rapidly. To create the plates for use in the screening robot we needed to develop a method for rapidly and gently placing a single (or several) fish into each well in an automated fashion. We have engineered a microfluidics device, which is capable of delivering fish into wells automatically. The device has a system of delivering a stream of water past a detector, which upon detection of a fish in the water stream, will divert the flow from a waste line into the well (FIG. 4).

The device has a 50 ml syringe which is filled with a high concentration of fish fry (at least 5 per ml) in tank water. The solution flows from the syringe at a moderate rate (typically 1 ml per 15 seconds). As fish enter the tubing connected to the syringe, their movement is retarded by the flow of liquid. As a fish passes a photointerrupter circuit, it triggers the device to shut a pinch valve (V1; FIG. 4), which the fish just passed, opening a valve (V2) from a second fed syringe that contains only tank water. This valve ensures that only one fish is in the feeder tube at a time.

As the first set of pinch valves is switching, a second set of down stream valves is switching. Prior to a fish's arrival, the efflux is directed to a waste container. Upon the detection of a fish in the water stream, the efflux stream is redirected to a well. Enough water is passed into the well to ensure that the fish has been deposited in the well. The amount of water deposited into the well is, therefore, tightly controlled so each well has the same depth of water. This is important for the electrical stimulation to be consistent.

Automatic Distribution of Compounds into Multiwell Plates

Commercially available apparatus to enable automatic distribution is available from Beckman Instruments, Caliper Technologies, Newport Industries, Thorlabs, Inc. and Robbins industries, Inc. This equipment would be used to distribute compounds into wells containing fish. After sufficient exposure, the animals are tested for the ability to generate seizure upon electrical stimulation. Successful seizure blockage is viewed as a potential anticonvulsant for use ultimately in man and animals.

Electrical Stimulation Methods

Without being bound to any mechanism of action, the present inventors provide the following description for the effect of electrical stimulation at the threshold value in order to produce an epileptic seizure in the fish

The Optimal Stimulation Paradigm

These include the type of waveform that should be applied to the well, i.e., sinusoidal, squarewave, bi- or monopolar, whether the stimuli should be current or voltage-limited and the shape (pulse width and duty cycle). Classically, both current and voltage-limited stimuli trains have been used with either square or sinusoidal waveforms.

Optimally, we have determined that constant current is preferably applied to each well. Towards this end we have produced arrays of high voltage constant current ICs (LR8) from Supertex, Inc. These circuits allow regulated constant current pulses to be given to wells. Bipolar current pulses are then directed to specific wells via digitally addressable and isolated high-voltage analog switches (HV209; Supertex, Inc.).

Drug Discovery and Drug Screening

The present invention provides reliable detection of test compounds that is significantly more versatile and robust than previous assay systems. Importantly, the present invention provides the ability to screen multiple compounds simultaneously by providing the ability induce seizure robotically and record their intensity via a computer.

Selectivity of Candidate Modulators

The in vivo methods described above also establish the selectivity of a candidate modulator. The present invention provides a rapid method of determining the specificity of the candidate modulator. For example, such a system provides for the first time the ability to rapidly profile large numbers of test chemicals for systematically evaluating the selectivity of a candidate modulator in a simple, miniaturized high throughput format.

The present invention can be better understood with reference to the accompanying examples, which are illustrative and should not be construed as being limiting of the scope of the invention.

The present invention has been described with particular reference to the preferred embodiments. It should be understood that the foregoing descriptions and examples are only illustrative of the invention. Various alternatives and modifications thereof can be devised by those skilled in the art without departing from the spirit and scope of the present invention. Accordingly, the present invention is intended to embrace all such alternatives, modifications, and variations that fall within the scope of the appended claims.

1-27. (canceled)

28. A high throughput screening system comprising:
a plurality of wells which form a multiwell tissue culture plate, each well being capable of containing one or more sample;
a laser for producing a laser beam of appropriate wavelength for applying a threshold pain stimulation to the sample to produce an aversive behavior response;
a plurality of photoemitters situated above or below each well of the multiwell tissue culture plate; and
an array of photodetectors situated below or above each well for recording said response to aversive behavior in real time;
wherein certain wells of the multiwell tissue culture plate can contain one or more chemical compounds which serve as candidate pharmacological agents for antagonizing nocioceptive behavior in the sample or ameliorating the nocioception.

29. The high throughput screening system of claim 28, wherein said laser can be focused on the epidermal surface of said sample with a focusing assembly.

30. The high throughput screening system of claim 29, wherein said focusing assembly is selected from the group consisting of: a lens, pinhole, and a combination thereof.

31. The high throughput screening system of claim 30, wherein said laser beam is not appreciably absorbed by water.

32. The high throughput screening system of claim 28, wherein said candidate pharmacological compound is an analgesic.

33. The high throughput screening system of claim 28, wherein said candidate pharmacological compounds are agents to treat neurological diseases.

34. A method of screening a candidate pharmacological agent, comprising the steps of:
placing said candidate pharmacological agent in a multiwell tissue culture plate having a plurality of wells, each well being capable of containing one or more sample, wherein certain wells of the multiwell tissue culture plate can contain one or more chemical compounds;
applying a stimulus onto said sample to produce a change in the behavior of said sample;
recording said change in behavior in real time to select candidates with superior performance.

35. (canceled)

36. The method of claim 34, wherein said stimulus is applied using a laser having a laser beam of appropriate wavelength for applying a threshold pain stimulation to the sample to produce an aversive behavior response in said sample.

37. The method of claim 34, wherein said change in behavior is recorded in real time using an array of photodetectors.

38. The method of claim 34, wherein said sample is a fish selected from the group consisting of: Medaka Fish (Orzyias latipes), Astronotus ocellatus, Danio rerio, Anguilla anguilla, Chelon labroses, Salmo trutta fario, Oncorhynchus mykiss, Oreochromis mossambicus, Eigenmannia virescens, Cyprinus carpio, Stephanolepis cirrhifer, Carassius auratus, Gosterosteus aculeatus, Clarias batrachus, Apteronotus leptorhynchus and a combination thereof.

39. The method of claim 34, wherein said candidate pharmacological agent is selected from the group consisting of:
a cardiac pharmacological agent, agents to treat neurological diseases, compounds which ameliorate abnormalities in the Q-T cardiac interval, analgesics, agents for antagonizing nocioceptive behavior, agents for ameliorating the nocioception, and a combination thereof.

40. The method of claim 34, wherein the steps of the method are carried out using a high throughput screening system having an illumination source, stimulation electrodes, a multiwell tissue culture plate, a photodetector, and an A/D converter.

41. The method of claim 34, wherein said stimulation electrodes are made of at least one material selected from the group consisting of:
gold, platinum, palladium, chromium, stainless steel alloy, molybdenum, iridium, tungsten, tantalum and titanium.

42. The method of claim 35, wherein said electrodes are separated by a gap of about 1 to 10 mm.
